(19) United States
(12) Patent Application Publication
(10) Pub. No.: US 2024/0404678 A1
(43) Pub. Date: Dec. 5, 2024

(54) RECOMMENDATION SYSTEM FOR A CONNECTED FITNESS PLATFORM

(71) Applicant: Peloton Interactive, Inc., New York, NY (US)

(72) Inventors: Shoya YOSHIDA, New York, NY (US); Nganba Meetei, New Providence, NJ (US)

(21) Appl. No.: 18/680,904

(22) Filed: May 31, 2024

Related U.S. Application Data
(60) Provisional application No. 63/505,517, filed on Jun. 1, 2023.

(51) Int. Cl. G16H 20/30; A63B 24/00; G06N 20/00

(52) U.S. Cl. CPC G16H 20/30; A63B 24/0075; G06N 20/00; A63B 2024/0068; A63B 2225/20

(57) ABSTRACT

Systems and methods that generate and perform class or content recommendations to users of a connected fitness platform are described. In some embodiments, the systems and methods may address a cold-start problem that often arises in connected fitness platforms, providing guidance to new users of the platform as they navigate through fitness content (of varying difficulty or experience levels) of the platform. The systems and methods introduce and adapt a transformer model to determine classes or content to recommend to new users of the platform.

### CROSS REFERENCE TO RELATED APPLICATIONS

This application claims priority to U.S. Provisional Patent Application No. 63/505,517, filed on Jun. 1, 2023, entitled RECOMMENDATION SYSTEM FOR A CONNECTED FITNESS PLATFORM, which is hereby incorporated by reference in its entirety.

### BACKGROUND

The world of connected fitness is an ever-expanding one. This world can include a user taking part in an activity (e.g., running, cycling, lifting weights, and so on), other users also performing the activity, and other users doing other activities. The users may be utilizing a fitness machine (e.g., a treadmill, a stationary bike, a strength machine, a stationary rower, and so on), or may be moving through the world on a bicycle or other equipment.

The users can also be performing other activities that do not include an associated machine, such as running, strength training, yoga, stretching, hiking, climbing, and so on. These users can have wearable devices or mobile devices (e.g., heart rate monitors) that monitor the activity or performance of the users. The users can also perform the activity in front of a user interface (e.g., a display or device) presenting content associated with the activity, or outside of any displayed content.

The user interface, whether a mobile device, a display device, or a display that is part of a machine, can provide or present interactive content to the users. For example, the user interface can present live or recorded classes, video tutorials of activities, leaderboards and other competitive or interactive features, progress indicators (e.g., via time, distance, and other metrics), and so on.

### BRIEF DESCRIPTION OF THE DRAWINGS

Embodiments of the present technology will be described and explained through the use of the accompanying drawings.

FIG. 1 is a block diagram illustrating a suitable network environment for users of an exercise system.

FIG. 2A is a block diagram illustrating example modules of a recommendation system.

FIG. 2B is a diagram illustrating an example context-augmented start token for training a machine learning model.

FIG. 3 is a diagram illustrating an example user interface of a connected fitness platform.

FIG. 4 is a diagram illustrating operations of a recommendation system.

FIG. 5 is a flow diagram illustrating a method for determining a recommendation to a user for an exercise class.

In the drawings, some components are not drawn to scale, and some components and/or operations can be separated into different blocks or combined into a single block for discussion of some of the implementations of the present technology. Moreover, while the technology is amenable to various modifications and alternative forms, specific implementations have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

### Overview

Various systems and methods that enhance an exercise activity performed by a user are described. In some embodiments, the systems and methods generate and perform class or content recommendations to users of a connected fitness platform.

For example, the systems and methods may address a cold-start problem that often arises in connected fitness platforms. As described herein, the cold-start problem relates to the platform attempting to provide recommendations for classes or other content to new users, without having knowledge about a user's preferences, skill level, activity level, fitness level, and so on.

Thus, the systems and methods may provide guidance to new users of the platform as they navigate through fitness content (of varying difficulty or experience levels) provided by the platform. In some embodiments, the systems and methods introduce a transformer model (e.g., a Fitness New user Experience Transformer or FitNEXT) model, which is an application of a transformer architecture to the cold-start problem of recommending content to users of the connected fitness platform.

Further, the systems and methods employ certain enhanced tokens when training the transformer model. For example, the systems and methods may generate and/or utilize a context-augmented start token (CAST) to represent the start of a user's fitness journey in a sequence of classes or content. Using the CAST, the systems and methods may train the transformer model using a next-item prediction task for data sets that include first portions (e.g., the first few classes) of workout histories associated with active users of the connected fitness platform.

Thus, the transformer model is trained to understand how user fitness journeys (e.g., sequences of classes) progress or change over time, and can determine and present class or content recommendations to beginner users that are appropriate to the characteristics and/or skill levels of the users, among other benefits.

Various embodiments of the system and methods will now be described. The following description provides specific details for a thorough understanding and an enabling description of these embodiments. One skilled in the art will understand, however, that these embodiments may be practiced without many of these details. Additionally, some well-known structures or functions may not be shown or described in detail, so as to avoid unnecessarily obscuring the relevant description of the various embodiments. The terminology used in the description presented below is intended to be interpreted in its broadest reasonable manner, even though it is being used in conjunction with a detailed description of certain specific embodiments.

### Examples of a Suitable Exercise Platform

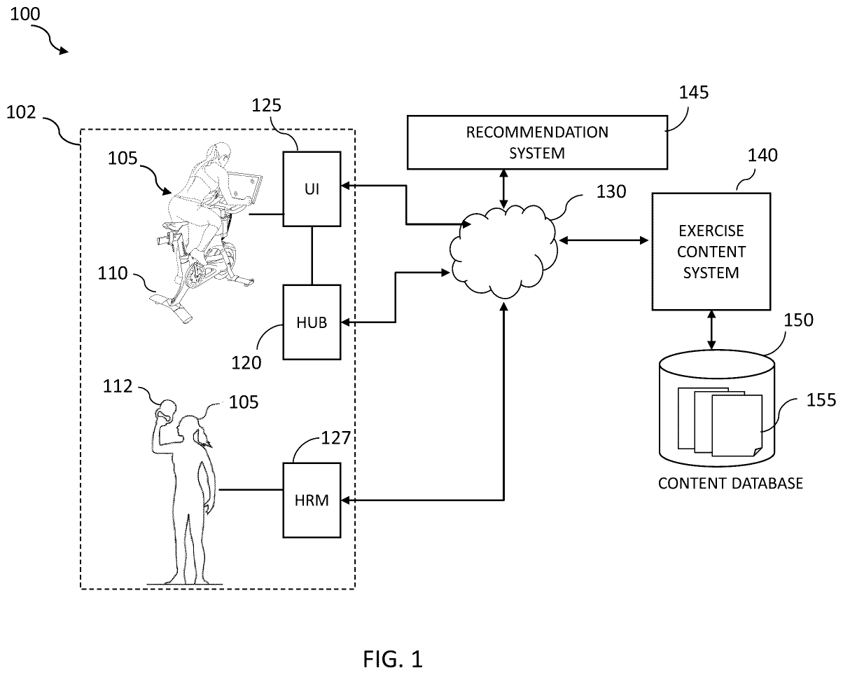

The technology described herein is directed, in some embodiments, to providing a user with an enhanced user experience (e.g., a personalize experience) when performing an exercise activity, such as an exercise activity as part of a connected fitness system or other exercise system. FIG. 1 is a block diagram illustrating a suitable network environment 100 for users of an exercise system.

The network environment 100 includes an activity environment 102, where a user 105 is performing an exercise activity, such as a cycling activity. In some cases, the user 105 can perform the activity with an exercise machine 110, such as exercise bicycle, a treadmill, a rowing machine, a stair climber, and so on. Further, the exercise activity performed by the user 105 can include a variety of different workouts, activities, actions, and/or movements not associated with a machine, such as movements associated with lifting weights 112 (as shown), stretching, doing yoga, pilates, rowing, running, cycling, jumping, sports movements (e.g., throwing a ball, pitching a ball, hitting, swinging a racket, swinging a golf club, kicking a ball, hitting a puck), and so on.

The exercise machine 110 can assist or facilitate the user 105 to perform the movements and/or can present interactive content to the user 105 when the user 105 performs the activity. For example, the exercise machine 110 can be a stationary bicycle, a stationary rower, a treadmill, a weight machine, or other machines. As another example, the exercise machine 110 can be a display device that presents content (e.g., streamed classes, dynamically changing video, audio, video games, instructional content, and so on) to the user 105 during an activity or workout.

The exercise machine 110 includes a media hub 120 and a user interface 125. The media hub 120, in some cases, captures images and/or video of the user 105, such as images of the user 105 performing different movements, or poses, during an activity. The media hub 120 can include a camera or cameras, a camera sensor or sensors, or other optical sensors configured to capture the images or video of the user 105.

In some cases, the media hub 120 includes components configured to present or display information to the user 105. For example, the media hub 120 can be part of a set-top box or other similar device that outputs signals to a display, such as the user interface 125. Thus, the media hub 120 can operate to both capture images of the user 105 during an activity, while also presenting content (e.g., time-based or distance-based experiences, streamed classes, workout statistics, and so on) to the user 105 during the activity.

The user interface 125 provides the user 105 with an interactive experience during the activity. For example, the user interface 125 can present user-selectable options that identify live classes available to the user 105, pre-recorded classes available to the user 105, historical activity information for the user 105, progress information for the user 105, instructional or tutorial information for the user 105, and other content (e.g., video, audio, images, text, and so on), that is associated with the user 105 and/or activities performed (or to be performed) by the user 105.

In some cases, a heart rate monitor (HRM) 127 or other wearable device (e.g., smart watch, headphones, fitness trackers, and so on) can capture biometric information about the user 105, such as heart rate, movement information, sleep information, and so on. The HRM 127 can capture the user's heart rate and other information during machine-based activities and/or other activities, such as offline or class-based activities that do not utilize the exercise machine 110. In some cases, the exercise machine can include components configured to capture biometric information for the user 105, such as heart rate information. Further, the HRM 127 or smart watch may also provide a user interface (e.g., the user interface 125) to the user.

The exercise machine 110, the media hub 120, and/or the user interface 125 can send or receive information over a network 130, such as a wireless network. Thus, in some cases, the user interface 125 is a display device (e.g., attached to the exercise machine 110), that receives content from (and sends information, such as user selections) an exercise content system 140 over the network 130. In other cases, the media hub 120 controls the communication of content to/from the exercise content system 140 over the network 130 and presents the content to the user via the user interface 125.

The exercise content system 140, located at one or more servers remote from the user 105, can include various content libraries (e.g., classes, movements, tutorials, and so on) and perform functions to stream or otherwise send content to the machine 110, the media hub 120, and/or the user interface 125 over the network 130.

A content database 150 stores content 155 (e.g., video files) that presents a pre-recorded class to a user. The content can include images, video, and other visual information that present the class, music and other audio information to be played during the activity, and various overlay or augmentation information that is presented along with the audio/video content. Further, the database 150 can include various content libraries (e.g., classes, movements, tutorials, and so on) associated with the content presented to the user during a selected experience.

As described herein, a recommendation system 145 can include various components configured to provide recommendations to users of a connected fitness platform. For example, the recommendation system 145 can include some or all aspects of the transformer model described herein. Further details regarding the functionality of the recommendation system 145 and various models will be described in the following sections.

FIG. 1 and the components, systems, servers, and devices depicted herein provide a general computing environment and network within which the technology described herein can be implemented. Further, the systems, methods, and techniques introduced here can be implemented as special-purpose hardware (for example, circuitry), as programmable circuitry appropriately programmed with software and/or firmware, or as a combination of special-purpose and programmable circuitry. Hence, implementations can include a machine-readable medium having stored thereon instructions which can be used to program a computer (or other electronic devices) to perform a process. The machine-readable medium can include, but is not limited to, floppy diskettes, optical discs, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other types of media/machine-readable medium suitable for storing electronic instructions.

The network or cloud 130 can be any network, ranging from a wired or wireless local area network (LAN), to a wired or wireless wide area network (WAN), to the Internet or some other public or private network, to a cellular (e.g., 4G, LTE, or 5G network), and so on. While the connections between the various devices and the network 130 and are shown as separate connections, these connections can be any kind of local, wide area, wired, or wireless network, public or private.

Further, any or all components depicted in the Figures described herein can be supported and/or implemented via one or more computing systems, services (e.g., cloud instances), or servers. Although not required, aspects of the various components or systems are described in the general context of computer-executable instructions, such as routines executed by a general-purpose computer, e.g., mobile device, a server computer, or personal computer. The system can be practiced with other communications, data processing, or computer system configurations, including: Internet appliances, hand-held devices, wearable devices, or mobile devices (e.g., smart phones, tablets, laptops, smart watches), all manner of cellular or mobile phones, multi-processor systems, microprocessor-based or programmable consumer electronics, set-top boxes, network PCs, mini-computers, mainframe computers, AR/VR devices, gaming devices, and the like. Indeed, the terms “computer,” “host,” and “host computer,” and “mobile device” and “handset” are generally used interchangeably herein and refer to any of the above devices and systems, as well as any data processor.

Aspects of the system can be embodied in a special purpose computing device or data processor that is specifically programmed, configured, or constructed to perform one or more of the computer-executable instructions explained in detail herein. Aspects of the system may also be practiced in distributed computing environments where tasks or modules are performed by remote processing devices, which are linked through a communications network, such as a Local Area Network (LAN), Wide Area Network (WAN), or the Internet. In a distributed computing environment, program modules may be located in both local and remote memory storage devices.

Aspects of the system may be stored or distributed on computer-readable media (e.g., physical and/or tangible non-transitory computer-readable storage media), including magnetically or optically readable computer discs, hard-wired or preprogrammed chips (e.g., EEPROM semiconductor chips), nanotechnology memory, or other data storage media. Indeed, computer implemented instructions, data structures, screen displays, and other data under aspects of the system may be distributed over the Internet or over other networks (including wireless networks), or they may be provided on any analog or digital network (packet switched, circuit switched, or other scheme). Portions of the system may reside on a server computer, while corresponding portions may reside on a client computer such as an exercise machine, display device, or mobile or portable device, and thus, while certain hardware platforms are described herein, aspects of the system are equally applicable to nodes on a network. In some cases, the mobile device or portable device may represent the server portion, while the server may represent the client portion.

### Examples of Providing Class/Content Recommendations to Users

As described herein, in some embodiments, the systems and methods perform various methods or operations to provide recommendations of classes and other content to users of a connected fitness platform.

As described herein, addressing the cold-start problem of generating personalized recommendations for new users can be useful for guiding them through a large corpus of unfamiliar digital content, enabling a connected fitness platform to ensure long-term user retention and satisfaction by providing users with engaging and appropriate content for their needs and/or experience levels. However, the platform typically has very limited information about new users.

For example, when a class to be recommended is an instructor-led workout class that varies in levels of difficulty (e.g., across different movements or sections), the cold-start problem introduces problems associated with introducing the new user to the class. Users start at different fitness and experience levels, which often evolve over time. For users who are fairly new to fitness, they necessitate a gradual progression from beginner classes to advanced classes.

However, for other users that are new to a platform but not new to certain exercise activities (e.g., they are experience runners, cyclists, or have trained with weights) a recommendation system should quickly adapt to their levels, and avoid recommendations (e.g., intro classes) that do not match the skill/experience levels of these new users. Thus, the platform may employ a recommendation system, such as the recommendation system 145, that addresses such problems when recommending content (e.g., exercise classes) for users new to the platform.

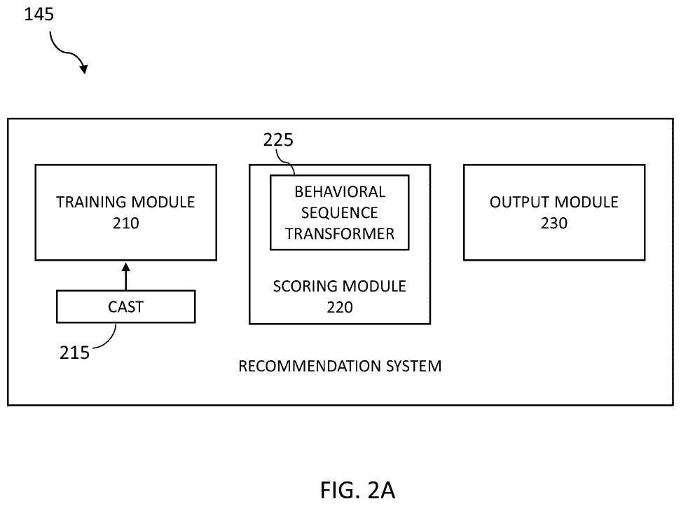

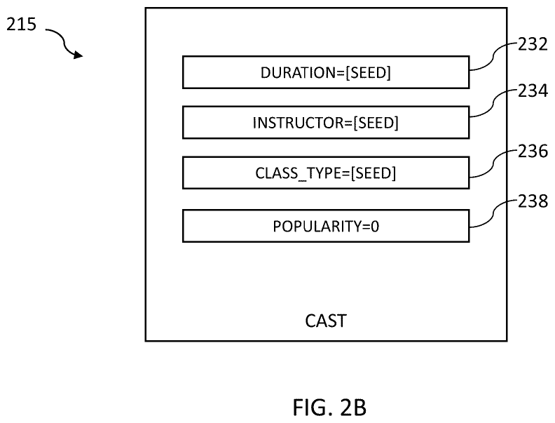

FIG. 2A is a block diagram illustrating example modules of the recommendation system 145. The modules or components of the recommendation system 145 can be implemented with a combination of software (e.g., executable instructions, or computer code) and hardware (e.g., at least a memory and processor). Accordingly, as used herein, in some embodiments, a component/module is a processor-implemented component/module and represents a computing device having a processor that is at least temporarily configured and/or programmed by executable instructions stored in memory to perform one or more of the functions that are described herein. The recommendation system 145, which may be part of a connected fitness system or platform, includes a training module 210, a scoring module 220, and an output module 230.

In some embodiments, the training module 210 is configured and/or programmed to train a transformer model using a context-augmented start token (CAST) 215 that represents a start state for the potential user within the connected fitness platform. The transformer model receives or access various input from the connected fitness system, such as user features and a sequence of workouts (e.g., a sequence of classes), where each workout is represented by contextually rich metadata about the workout and/or the taken class. In some cases, the training data leverages beginning portions (e.g., a first few classes) of for workout sequences performed by active users if the platform. Thus, the model may learn behaviors of many different users when they first joined the platform and use the learning when generating appropriate recommendations for current or future new members (e.g., of which little is known by the platform).

The training module 210, in some embodiments, trains a behavioral sequence transformer (BST) to expand upon the BST model and apply it to the cold-start scenarios described herein, adapting to user preferences and levels as they perform activities within the platform. The training module 210 may employ and utilize a start token, such as the CAST 215, which includes seed values for multiple metadata types associated with exercise classes provided by the connected fitness platform. The training module 210 may trains the BST using a next item prediction task and an initial workout sequence represented by the CAST.

In some cases, the training module 210 trains the transformer model on a new user data set, utilizing the CAST 215 to mark or represent a beginning of a user's fitness journey within the platform. In doing so, the model may learn a user's start state.

The training module 210 may construct training data D=[H₁,H₂, . . . H_{N}], where Hᵢ is the beginning portion of the workout history of user i and N is the number of active users on the platform. Specifically, Hᵢ=[W_{i,1}, W_{i,2} . . . , W_{i,p}], where W_{i,j} is the j-th workout completed by user i using the first P workouts from each user (e.g., P=10). Each workout W_{i,j} is denoted as a feature vector W_{i,j}=[Sᵢ; C_{i,j}; Rₖ], where Sᵢ is static user features that stays constant throughout the sequence, C_{i,j} is contextual features about the workout for user i on their j-th workout, and Rₖ is the metadata features for the k-th class. Each of these feature vectors are represented by s, c, or r number of attributes, respectively; for example Rₖ={A_{Rk,1}, A_{Rk,2}, . . . , A_{Rk,r}} where each A_{Rk,a} is some feature attribute, such as the instructor or the class type.

The training module 210 also prepends the CAST 215, denoted by T₀, at the beginning of each training sequence, such that H′ᵢ=[T₀; W_{i,1}, W_{i,2} . . . , W_{i,P}]. As described herein, the CAST 215 is start token for the recommendation system 145, and represented by metadata and augmented with contextual information.

FIG. 2B depicts features of the CAST 215. The CAST 215 includes a same list of features used for each of the items, and for each categorical variable the CAST 215 receives a special unique value. The CAST 215 acts as a separate unique representation to explicitly mark the beginning of a fitness journey for a user, enabling the transformer model to learn how each feature relates to new user behaviors. For example, the transformer model may learn that an embedding vector for a [SEED] is more similar to an embedding vector for a relatively easier 20-minute class (e.g., suitable for new users) than a harder 60-minute class in the embedding table for the duration of the class.

The CAST 215, therefore, can include various types of metadata, such as Duration=[SEED] 232, Instructor=[SEED] 234, Class_type=[SEED] 236 and/or Popularity=0 238. Using the features of the CAST 215, the transformer model may capture how a typical new user fitness journey begins, enabling the model to guide new users through a recommended path of workouts within the connected fitness platform.

In some cases, while no classes are the same, the platform may create and provide several new instructor-led workout classes on a daily or weekly basis. Thus, classes with similar metadata can and will re-occur. For example, the platform may release several beginner friendly cycling cases each week, which share the class type attribute of “beginner.” The training module 210 may thus learn from historical workout history and apply the learning when generating recommendations for new users.

In some embodiments, the training module 210 may train the transformer model using a next item prediction task or operation, where the model predicts an item that a user may interact in a next instance, given previous interactions or events of the user. For example, for user i and target class Rₙ, the probability of the user converting on the target class can be modeled as P(Rₙ|[H_{i,j<n}; θ), where θ denotes the model parameters. In some cases, such as when training the model predict a first workout class for a new user, the workout sequence may be one item, represented by the CAST 215. Thus, the transformer model may learn about first best classes that new beginners typically take on a feature-level, adjusting any embeddings learned for each feature of the CAST 215.

In some embodiments, the scoring module 220 is configured and/or programmed to generate a recommendation score for a target exercise class with respect to a potential user of the target exercise class. The scoring module 220 may receiver user characteristics and workout sequence data for the potential user, apply a transformer model (e.g., a BST 225) to the user characteristics and workout sequence data, and generate the recommendation score based on a comparison of an embedding/encoding generated by applying the BST to the user characteristics and the workout sequence data and an embedding/encoding associated with the target exercise class.

In some cases, the scoring module 220 may generate the recommendation score for the target exercise class by performing a dot product operation on the embedding/encoding generated by applying the BST to the user characteristics and the workout sequence data and the embedding/encoding associated with the target exercise class to determine a similarity.

In some cases, the scoring module 220, via the BST 225, utilizes a multi-layer perceptron (MLP) to encode the embedding from the user characteristics and the workout sequence data and/or to encode the embedding associated with the target exercise class. Further details regarding operation of the BST 225 within the recommendation system 145 are described herein.

In some embodiments, the output module 230 is configured and/or programmed to present a recommendation to the potential user for the target exercise class when the recommendation score is above a threshold score. For example, the output module 230 may present the recommendation to the potential user for the target exercise class via a user interface associated with an exercise machine upon which the potential user views exercise classes streamed by the connected fitness platform.

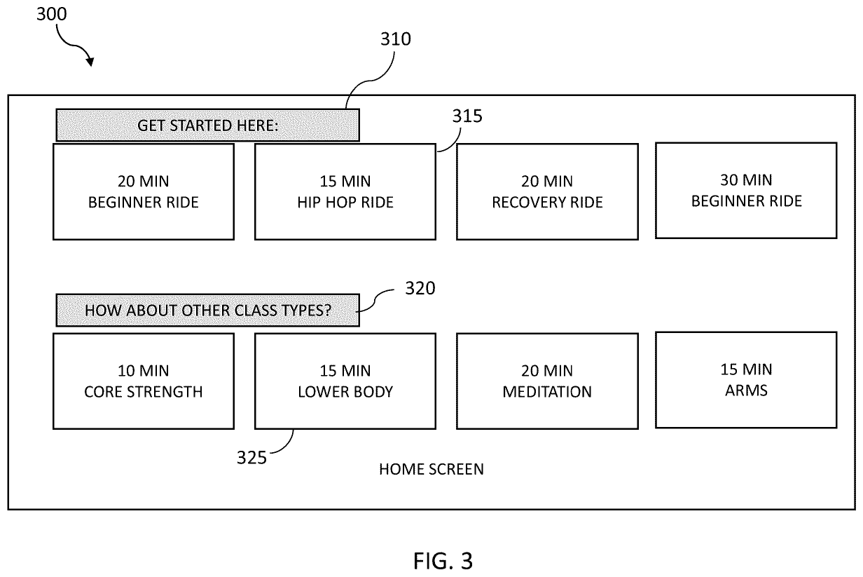

FIG. 3 depicts an example user interface 300 of a connected fitness platform, such as a UI presented via a display (e.g., a home screen) associated with an exercise machine (e.g., exercise bicycle, treadmill, and so on), a mobile application supported by a mobile device of the user, and so on.

For example, the UI 300 may be a home screen presented by the connected fitness platform, and include multiple rows 310, 320 of user-selectable and recommended classes 315, 320. The output module 230 may interact with the UI 300, causing the UI 300 to presented recommended classes to a new user. As shown, the recommended classes 315, 325 may be targeted to the new user based on operations of the recommendation system 145 and include classes that enable a new user to “get started” in one modality and/or include classes that introduce the new user to “other class types” or modalities provided by the platform.

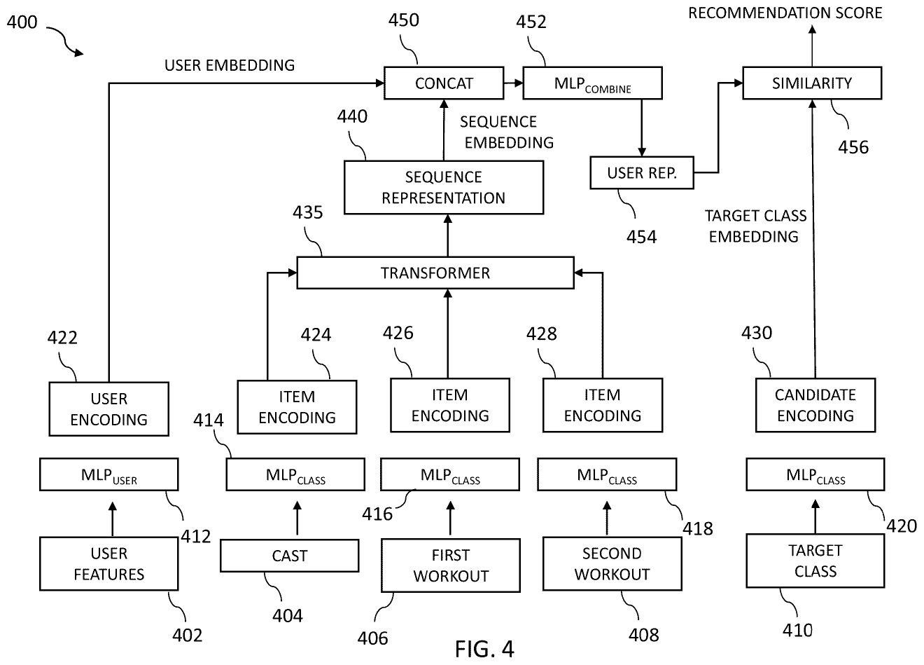

As described herein, the recommendation system 145 utilizes the BST 225 to determine scores for target exercise classes and generate recommendations of classes or other content to users based on the scores. FIG. 4 is a diagram illustrating operations 400 of the recommendation system 145.

Adapting the BST 225, the recommendation system 145 utilizes various encoders, such as specific MLPs, to generate embeddings from user and class data. A user feature encoder, or MLP_{USER} 412, encodes and/or embeds static user features 402 to generate a user encoding 422 (or user embedding). The MLP_{USER} 412 may encode various user features, such as demographic or characteristic information (e.g., language, country, and so on) or other non-sequential or activity user information.

A sequence encoder is a transformer encoder 435, which captures a sequence representation 440, or sequence embedding/encoding, using item encodings 424, 426, 428 generated by class or item encoders, such as MLP_{CLASS} 414, 416, 418. The transformer encoder 435 receives the item encodings 424, 426, 428 and pools received encodings to generate the sequence representation 440. As described herein, a workout item may be a specific workout (e.g., a first workout 406, a second workout 408), and/or represented by a CAST 404, such as the CAST 215.

A target class encoder also uses a class or item encoder, such as MLP_{CLASS} 420. The MLP_{CLASS} 420 receives a target class item 410 and generates a candidate encoding 430 or target class embedding.

A concatenation module 450 concatenates the user embedding and the sequence embedding, which is then input into another encoder, named MLP_{COMBINE} 452, to generate a user representation 454 that combines the user and sequence embeddings. A similarity module 456 determines a similarity between the user representation 454 and the target class embedding to generate a recommendation score for the target class 410.

For example, the similarity module 456 may perform a dot product operation to the received embeddings/encodings, which measures the similarity (e.g., cosine similarity) between the embeddings/encodings (e.g., represented as vectors). In some cases, a sigmoid function receives the cosine similarity and generates a final recommendation score for the target class 410. The recommendation system 145 may then compare the score to scores of other classes and/or a threshold score and determine whether to recommend the target class 410 to the user based on the comparison.

As described herein, the recommendation system 145 may perform various processes, operations, and/or methods when generate recommendations of classes to users of a connected fitness platform. For example, the system 145 may employ various methods for generating a recommendation score for a target exercise class with respect to a potential user of the target exercise class.

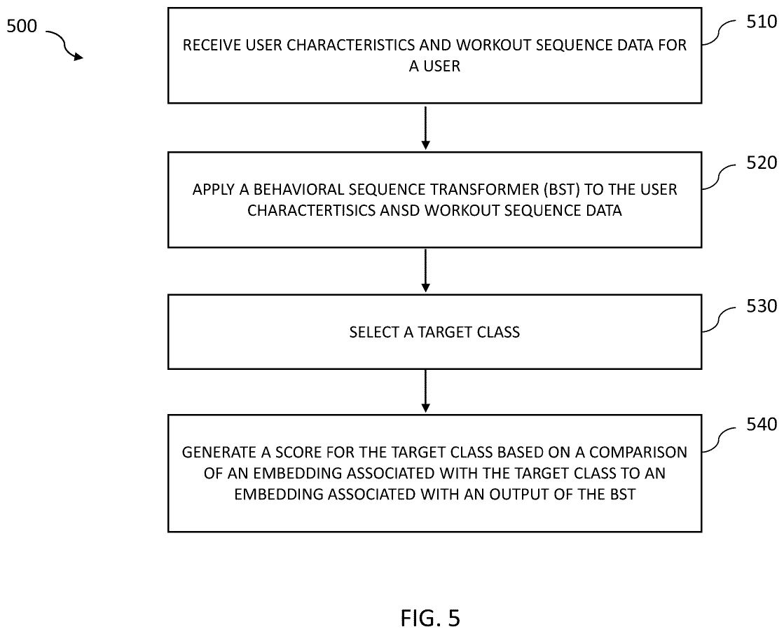

FIG. 5 is a flow diagram illustrating a method 500 for determining a recommendation to a user for an exercise class. The method 500 may be performed by the recommendation system 145 and, accordingly, is described herein merely by way of reference thereto. It will be appreciated that the method 500 may be performed on any suitable hardware.

In operation 510, the recommendation system 145 receives user characteristics and workout sequence data for the potential user. For example, the scoring module 220 may receive data that identifies user characteristics or demographics, as well as metadata associated with an initial sequence of workouts performed by the user, and/or a start token (e.g., the CAST 215), which represents a start state for the user within the platform.

In operation 520, the recommendation system 145 applies a behavioral sequence transformer (BST) to the user characteristics and workout sequence data. For example, the scoring module 220 utilizes a multi-layer perceptron (MLP) to encode embeddings representing the user data and sequence data.

In operation 530, the recommendation system 145 selects a target class form a set of available classes to the user, and, in operation 540, generates the recommendation score based on a comparison of an embedding generated by applying the BST to the user characteristics and the workout sequence data and an embedding associated with the target exercise class. For example, the scoring module 220 may generate the recommendation score for the target exercise class by performing a dot product operation on the embedding generated by applying the BST to the user characteristics and the workout sequence data and the embedding associated with the target exercise class to determine a similarity.

The recommendation system 145, via the output module 230, may then present a recommendation to the potential user for the target exercise class when the recommendation score is above a threshold score. For example, the output module 230 may present the recommendation to the potential user for the target exercise class via a user interface (e.g., a home screen) associated with an exercise machine upon which the potential user views exercise classes streamed by the connected fitness platform.

Thus, the system 145 may train a behavioral sequence transformer (BST) using a context-augmented start token (CAST) that represents a start state for a new user within a connected fitness platform, apply the trained BST to user characteristics and workout sequence data associated with the new user, generate, for one or multiple exercise classes available to the new user via the connected fitness platform, a recommendation score based on a comparison of an embedding generated by applying the BST to the user characteristics and the workout sequence data and an embedding associated with the multiple exercise classes, and present recommendations to the new user for a subset of exercise classes of the multiple exercise classes on the generated recommendation scores.

In doing so, the connected fitness platform can employ or utilize an approach of applying a transformer architecture towards the cold-start problem in connected fitness platforms and provide targeted or enhanced class/content recommendations to new users of the platform, among other benefits.

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or”, in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further embodiments of the disclosure.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the technology may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

From the foregoing, it will be appreciated that specific embodiments have been described herein for purposes of illustration, but that various modifications may be made without deviating from the spirit and scope of the embodiments. Accordingly, the embodiments are not limited except as by the appended claims.

What is claimed is:

1. A recommendation system for a connected fitness platform, the recommendation system comprising:
a scoring module that is configured to generate a recommendation score for a target exercise class with respect to a potential user of the target exercise class by:
receiving user characteristics and workout sequence data for the potential user;
applying a behavioral sequence transformer (BST) to the user characteristics and workout sequence data; and
generating the recommendation score based on a comparison of an embedding generated by applying the BST to the user characteristics and the workout sequence data and an embedding associated with the target exercise class; and
an output module that is configured to present a recommendation to the potential user for the target exercise class when the recommendation score is above a threshold score.

2. The recommendation system of claim 1, further comprising:
a training module that is configured to train the BST using a context-augmented start token (CAST) that represents a start state for the potential user within the connected fitness platform.

3. The recommendation system of claim 2, wherein the CAST includes seed values for multiple metadata types associated with exercise classes provided by the connected fitness platform.

4. The recommendation system of claim 2, wherein the training module trains the BST using a next item prediction task and an initial workout sequence represented by the CAST.

5. The recommendation system of claim 1, wherein the scoring module generates the recommendation score for the target exercise class by performing a dot product operation on the embedding generated by applying the BST to the user characteristics and the workout sequence data and the embedding associated with the target exercise class to determine a similarity.

6. The recommendation system of claim 1, wherein the scoring module utilizes a multi-layer perceptron (MLP) to encode the embedding generated by applying the BST to the user characteristics and the workout sequence data and to encode the embedding associated with the target exercise class.

7. The recommendation system of claim 1, wherein the output module presents the recommendation to the potential user for the target exercise class via a user interface associated with an exercise machine upon which the potential user views exercise classes streamed by the connected fitness platform.

8. The recommendation system of claim 1, wherein the output module presents the recommendation to the potential user for the target exercise class via a mobile application via which the potential user views exercise classes streamed by the connected fitness platform.

9. The recommendation system of claim 1, wherein the output module presents the recommendation to the potential user for the target exercise class via a home screen displayed by an exercise machine upon which the potential user views exercise classes streamed by the connected fitness platform.

10. The recommendation system of claim 1, wherein the output module presents the recommendation to the potential user for the target exercise class as a row of similar exercise classes via a home screen displayed by an exercise machine upon which the potential user views exercise classes streamed by the connected fitness platform.

11. A method performed by a recommendation system of a connected fitness platform, the method comprising:
generating a recommendation score for a target exercise class with respect to a potential user of the target exercise class by:
receiving user characteristics and workout sequence data for the potential user;
applying a behavioral sequence transformer (BST) to the user characteristics and workout sequence data; and
generating the recommendation score based on a comparison of an embedding generated by applying the BST to the user characteristics and the workout sequence data and an embedding associated with the target exercise class; and
presenting a recommendation to the potential user for the target exercise class when the recommendation score is above a threshold score.

12. The method of claim 11, further comprising:
training the BST using a context-augmented start token (CAST) that represents a start state for the potential user within the connected fitness platform.

13. The method of claim 12, wherein the CAST includes seed values for multiple metadata types associated with exercise classes provided by the connected fitness platform.

14. The method of claim 12, wherein training the BST includes training the BST using a next item prediction task and an initial workout sequence represented by the CAST.

15. The method of claim 11, wherein generating the recommendation score for the target exercise class includes performing a dot product operation on the embedding generated by applying the BST to the user characteristics and the workout sequence data and the embedding associated with the target exercise class to determine a similarity.

16. The method of claim 11, further comprising:
encoding the embedding generated by applying the BST to the user characteristics and the workout sequence data and the embedding associated with the target exercise class via a multi-layer perceptron (MLP).

17. The method of claim 11, wherein presenting the recommendation to the potential user for the target exercise class includes presenting the recommendation via a user interface associated with an exercise machine upon which the potential user views exercise classes streamed by the connected fitness platform.

18. The recommendation system of claim 1, wherein presenting the recommendation to the potential user for the target exercise class includes presenting the recommendation via a mobile application via which the potential user views exercise classes streamed by the connected fitness platform.

19. The recommendation system of claim 1, wherein presenting the recommendation to the potential user for the target exercise class includes presenting the recommendation via a home screen displayed by an exercise machine upon which the potential user views exercise classes streamed by the connected fitness platform.

20. A non-transitory, computer-readable medium whose contents, when executed by a computing system, cause the computing system to perform a method, the method comprising:
training a behavioral sequence transformer (BST) using a context-augmented start token (CAST) that represents a start state for a new user within a connected fitness platform;
applying the trained BST to user characteristics and workout sequence data associated with the new user;
generating, for multiple exercise classes available to the new user via the connected fitness platform, a recommendation score based on a comparison of an embedding generated by applying the BST to the user characteristics and the workout sequence data and an embedding associated with the multiple exercise classes; and
presenting recommendations to the new user for a subset of exercise classes of the multiple exercise classes on the generated recommendation scores.
